(19) United States
(12) Patent Application Publication
(10) Pub. No.: US 2003/0233365 A1
(43) Pub. Date: Dec. 18, 2003

(54) System and method for semantics driven data processing

(75) Inventors: John C. Schmit, Austin, TX (US); Harsh W. Sharma, Somerset, NJ (US)

Correspondence Address: CHALKER FLORES, LLP, 12700 PARK CENTRAL, STE. 455, DALLAS, TX, 75251, US

(73) Assignee: Metainformatics, Austin, TX

(21) Appl. No.: 10/412,663

(22) Filed: Apr. 11, 2003

Related U.S. Application Data
(60) Provisional application No. 60/372,274, filed on Apr. 12, 2002.

(51) Int. Cl. G06F 7/00

(52) U.S. Cl. 707/100

(57) ABSTRACT

The present invention provides a system, method and computer program for metadata conduit driven data integration in which data from one or more data sources is integrated using a pre-processor, a modeler, a metadata repository, a virtual data access engine and a web portal, wherein an integration server consumes the metadata stored in the repository to direct queries to data sources and aggregate data and provide functional views of this data to the information consumers. The metadata stored in the repository also drives generation of platform independent applications used in the life sciences domain (research and/or drug development and diagnostics).

### PRIORITY CLAIM

This application claims priority to U.S. Provisional Patent Application Serial No. 60/372,274, filed Apr. 12, 2002.

### TECHNICAL FIELD OF THE INVENTION

The present invention relates in general to the field of computer technology, and more particularly, to collecting, categorizing, integrating and analyzing any amount of heterogeneous metadata, both from internally generated sources and externally acquired sources, especially as it relates to life science data.

### BACKGROUND OF THE INVENTION

Without limiting the scope of the invention, its background is described in connection with life science metadata collection, analysis, integration, and processing, as an example.

Heretofore, in this field, businesses and companies, especially those involved in research and drug development within the life sciences industry, face a crisis due to rapid increases in semantic inconsistency/inaccuracy, volume and heterogeneity of data. Data generation resulting from faster, improved experimental apparatus and the improved methods and processes used for experimentation is now outpacing the ability to analyze the data. This leads to delays in data delivery and the outcomes they produce.

Since the completion of the Human Genome Project in 2000, the amount of data available to researchers about our genetic makeup and the associated data related to discovering new drugs has grown exponentially. The data volumes that pharmaceuticals and biotech's must deal with are now exceeding the petabyte threshold (10¹⁵). Unfortunately, access to this avalanche of data is of no use to researchers unless there is a way to quickly and effectively integrate the data into the formats they need. It is only after the quick and effective data integration that the data may then be supplied to specialized applications that will help identify possible new hypotheses or improvements, for example, new drugs, tests and screening methods. Any delay in the discovery and development of potential new drugs results in huge costs for both the companies and consumers where the estimated cost to develop a new drug is about $880 million and consumes 10-12 years of effort, the attrition rate of novel drugs at clinical phase III is about 45%. It has been estimated that the average amount that could be saved by eliminating one in 10 drug targets from research is $200 million. In addition, the estimated savings if there was a properly implemented and integrated data system would be at least $300 million for a large research and development company.

In the present marketplace, data integration and data management are key to successfully deriving value from data and for keeping a business as a leader in its industry. New, innovative techniques must be devised so that data analysis can stay in pace with the rate of data generation.

Current products that provide some data integration offer service that is both very slow (in near real-time or real-time), not compatible across platforms (too specialized for only one type of data), and not always user-friendly. Currently lacking, is a single product/service that integrates any type of life sciences data that arises from multiple sources as well as addresses semantic heterogenity of data and facilitates development of Life Sciences applications that can consume industry standard metadata. A system that offers this capability (or automation) should be both cost effective and improve the time-to-market of potential new market ideas such as, for example, drugs. In addition, there is a need to provide ease of use, such as through user-friendly software, for persons to access the data, store the data, re-analyze the data, create output files, and/or integrate multiple data sources in near real-time or real-time. Such user-friendly software will provide cost-savings for the business as well as the researcher/other persons involved in drug development and reduce time and effort that is now spent trying to manage cumbersome amounts of data from multiple businesses and/or other sources often leading to incorrect interpretations/decisions.

### SUMMARY OF THE INVENTION

There is a need to reduce the time, effort, and cost currently required to sift through unmanageable amounts of disparate data, data that is often isolated and from incompatible data sources. Currently, there is no near real-time or real-time access between persons and the multiple sources of data they need to access for research and drug development. With the present invention, data relevant to experimentation for research and/or drug development will be made accessible via metadata driven web services. In addition, scientific instruments will be able to consume the same metadata (embedded metadata) to drive data exchange among each other, potentially resulting in speedier drug discovery/development process. Furthermore, this invention will enable all persons involved in the research and drug development effort to share and understand semantically accurate information to make better decisions. Not only existing software applications and systems will benefit by tapping into the same semantics repertoire, but also new applications/system development will also be driven from the Model Driven Architecture principle that forms the cornerstone (and is endorsed by leading software standards organizations) of this invention. Another unique capability this invention will facilitate is unique identification of life sciences information assets (genes, proteins for example) by assigning industry standard ‘Unique Identifiers’ across the data repositories. This is an important feature of the ‘Virtual Data Integration’ capability of this invention. The benefit of the present invention is its ability to enable humans and machines involved understand and exchange the metadata using the same ‘Lingua Franca’—universal language—and cross-fertilize with all business platforms and technologies, regardless of type of data as long as the data source is computational or stored as bytes of information.

One form of the present invention is a metadata conduit driven software for integrating and analyzing life sciences data from one or more data sources comprising a modeler, a metadata repository, a virtual data access/integration engine, a portal and adapters for disparate data sources, wherein an integration server consumes the metadata stored in the repository to direct queries to data sources, aggregates data and provides functional views of this data to information consumers.

Another form of the present invention is the ability to embed components of the metadata into the instrumentation (hardware) involved in research/drug development (e.g., High Throughput Screening (“HTS”), Mass Spectrometry and other diagnostics instruments for drug discovery) and enable exchange of the output data using XML. This capability can be further enhanced by developing alert mechanisms to inform persons involved in drug development of results of interest in near real-time or real-time, potentially speeding up the discovery process.

The present invention may also be used for providing subscription based web services to one or more businesses and/or companies that require data integration. An example would be a Patent Filing Web Service that automates the process of preparing and filing patents. Using these web services, businesses/companies may work independently, accessing only specific data sources as needed, or may be combined to allow access to several independent data sources, including each others data sources.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a block diagram of a system in accordance with one embodiment of the present invention;

FIG. 2 is a block diagram of a system in accordance with another embodiment of the present invention;

FIG. 3 is a flow chart of a method in accordance with one embodiment of the present invention;

FIG. 4 is a block diagram of a system in accordance with another embodiment of the present invention;

FIG. 5 is a flow chart of a method in accordance with another embodiment of the present invention;

FIG. 6 is a screen shot of a MetaLife Modeler in accordance with one embodiment of the present invention;

FIG. 7 is a block diagram of a MetaLife Integration Server in accordance with one embodiment of the present invention;

FIG. 8 is a block diagram of a system in accordance with another embodiment of the present invention;

FIG. 9 is a diagram illustrating the uses of the MetaLife Modeler in accordance with one embodiment of the present invention;

FIG. 10 is a MetaModel for a BioAssay in accordance with one embodiment of the present invention;

FIG. 11 is a MetaModel for an ArrayDesign in accordance with another embodiment of the present invention;

FIG. 12 is a block diagram of a data flow in accordance with one embodiment of the present invention;

FIG. 13 is a block diagram of a system in accordance with another embodiment of the present invention;

FIG. 14 is a block diagram of a MetaLife Integration Server in accordance with another embodiment of the present invention;

FIG. 15 is a block diagram of a data flow in accordance with another embodiment of the present invention;

FIG. 16 is a block diagram of a system in accordance with another embodiment of the present invention;

FIG. 17 is a block diagram of a system in accordance with another embodiment of the present invention; and

FIG. 18 is a block diagram of a system in accordance with another embodiment of the present invention.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The system of the present invention represents a revolutionary advance for the most critical portion of a business—the data that drives it. Under the current systems used by many businesses, for example, businesses in the life sciences industry—in order to investigate a single drug candidate—a researcher and other persons involved might be required to examine several different databases many times over, each database housing different types of data such as genetic, proteomic, bibliographic, and patent information, often using separate software applications to address each database. This approach is not only time-consuming (searching for the same answer many times over) but prevents near real-time or real-time access to constantly expanding biological, proteomic and chemistry databases, since researchers must collect, reformat, and assimilate the continuous worldwide production of new life sciences data, and republish their databases at frequent intervals.

In contrast, the present invention will enable access to all current and historic data sources relevant to scientific investigations focused on drug development from a single, browser-based interface. By using web services and a metadata management repository, the present invention mediates near real-time or real-time access between one or more persons and the multiple data sources they need to access. Metadata is data about the content, quality, condition, and other characteristics of data. By making use of the latest web services technology to update the user interface automatically, the present invention informs users that new life science databases have entered the application service. Thus, the present invention provides a significantly improved method for those persons attempting to analyze isolated, incompatible data sources. And by freeing a person from the tedious and time-consuming task of data integration and updates, the present invention saves businesses and/or whole industries time and money as well as freeing up the employees from time-consuming data analysis allowing them to focus on their real work.

The present invention solves some of the current problems by providing a person or business a way to quickly and effectively integrate their data (from one or more sources) into ‘functional views’ they need. These functional views can be supplied to specialized applications that will help them identify possible candidates for new drugs and rapidly test those hypotheses. The present invention also offers solutions that process this data without always requiring the presence of one or more persons. In addition, the present invention is able to leverage components that a person and/or business is already utilizing because it is a hybrid model that insures that not only the person or business is satisfied with the software but that it is part of an integrated solution that interfaces with person's/business' already existing system(s).

The present invention, also referred to as ‘MetaNome™’, is a novel industry standards-based, scalable, platform independent repertoire of authentic semantics and business rules for the life sciences industry that aims to streamline the costly drug development process and enhance competitive edge. MetaNome is also a novel, industry standards-based, scalable, platform independent, horizontal metadata conduit for the life sciences industry that is understood by humans and machines to facilitate the understanding and integration of enterprise assets.

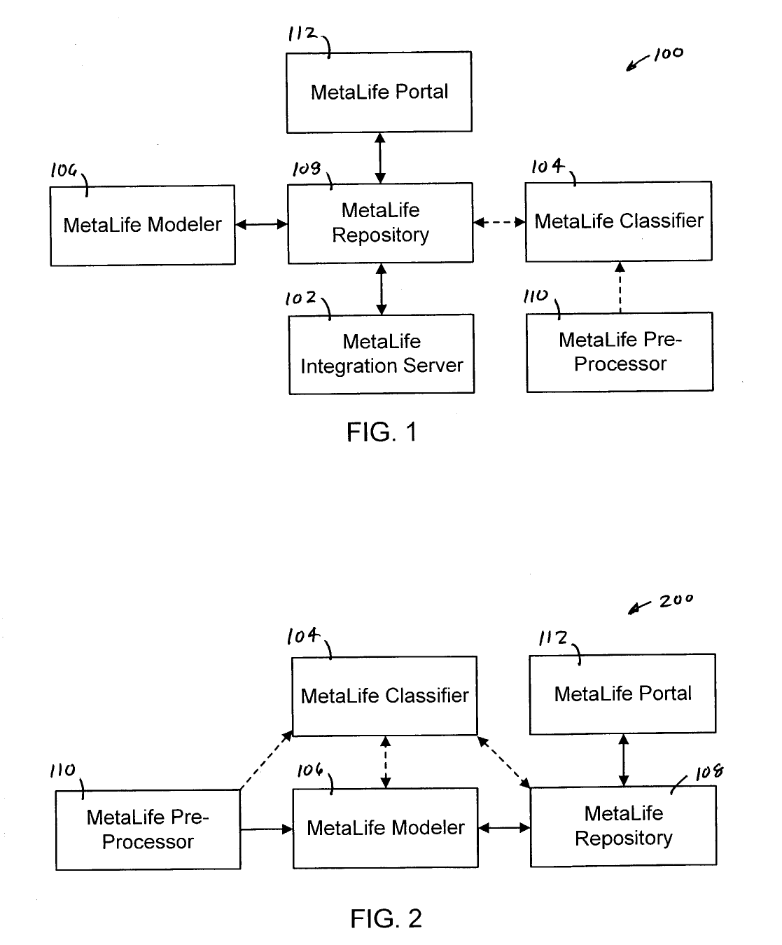

FIG. 1 is a block diagram of a system 100 in accordance with one embodiment of the present invention. The system 100 includes a MetaLife Integration Server 102, a MetaLife Classifier 104, a MetaLife Modeler 106, a MetaLife Repository 108, a MetaLife Pre-Processor 110 and a MetaLife Portal 112. The MetaLife Repository 108 is communicably coupled to the MetaLife Integration Server 102, the MetaLife Classifier 104 (optional), the MetaLife Modeler 106 and the MetaLife Portal 112. The MetaLife Classifier 104 is also communicably coupled to the MetaLife Pre-Processor 110 (optional). The dashed lines between the MetaLife Classifier 104 and the MetaLife Repository 108 and the MetaLife Pre-Processor 110 indicate that the MetaLife Classifier 104 and the MetaLife Pre-Processor 110 are optional. The MetaLife integration Server 102 provides run-time execution of Metadata for data integration and web services. The MetaLife Classifier 104 provides an additional capability to classify the metadata into functional views. The functional views can be output from the MetaLife Classifier 104, built manually in the MetaLife Modeler 106 and accessed from the MetaLife Repository 108. The MetaLife Modeler 106 is used to design MetaModels, PIMs, PSMs, XML Schemas and Web Services. The MetaLife Repository 108 stores MetaModels, PIMs/PSMs, Web Services' definitions and XML Schemas, SOAP, WSDL and UDDI, etc. The MetaModels may include CWM, MOF and UML. The PIMs/PSMs may include gene expression, genomeMaps, ChemInformatics, BioMolecular Sequence Analysis, Clinical Image Access Service, etc. The Web Service can be internal or external and may include Search GenBank, SearchMed, SearchProt and Patent Filing, etc. The MetaLife Pre-Processor 110 gathers, maps and integrates Metadata from various metadata sources. The MetaLife Portal 112 provides browser-based ‘views and reports’ of MetaLife repository components and metadata updates.

The Metadata Repository Models/Metamodels serves as the central hub into which a Virtual Data Access Engine, XML DTDs/Schemas, UDDI Repository and Adapters flow. Clinical Trials Data Repositories, Genomic Databases, Chemical Databases, Proteomics Databanks, Lab Instruments, Flat Files, XML/HTML Documents are examples of data sources that may all or independently flow into the Adapters. Flow is in either direction between the Metadata Repository Models, Metamodels and one or all of the following components: ETL Engine, Transform, UDDI Repository, XML, DTDs/Schemas, Virtual Data Access Engine. From the ETL Engine and the Virtual Data Access Engine flow may go to an Integrated Data Layer and Portal or web services. And, from the latter, the destinations may include one or more Web browsers, PC applications, Visalization Applications, and Wireless Devices. Users of the System include Administrators, Lab Technicians, Researchers, Chemists, Clinical Research Organizations, Proteomics Specialists, businesses and any other person requiring access to the system.

An important aspect of the system of the present invention involves the use of metadata management tools. Metadata is the primary means by which interoperability is achieved in a heterogeneous environment. Although interoperability is essentially facilitated by standard API's, it ultimately depends upon shared metadata as the definitions of systems' semantics and capabilities. Therefore, the capability to gather, store and publish application and system-level metadata is a ‘must have.’ Applications, tools, databases, and other components expose and discover metadata to enable cross-talk.

The system of the present invention includes data management software that will vastly simply the task of categorizing, integrating and analyzing the vast amounts of heterogeneous data, both from internally generated sources as well external life sciences research data. The present invention will remove the data integration and analysis burden from researchers and allow them to focus their efforts on research and development.

The present invention solves the following design challenges with the development of the present invention: Standardization of diverse interpretations of data (often same or regional flavors or based on business rules) resolved by creating a metadata repository that will manage metadata as well as directory of services (UDDI) that differentiates the present invention from others; and establishing the common Lingua Franca (common language) and ATM (Adapter-translation Mechanism) that allows standard format for data exchange and transformation resolved by the use of XML and ATM hubs.

The present invention may include of one or more of the following software components: MetaLife Pre-processor, MetaLife Classifier, MetaLife Modeler, MetaLife Repository; Virtual Data Access Engine; Portal, ETL Engine (Extract, Transformation & Load) and Adapters for various data sources. The components are discussed below.

The ETL Engine may include one of several commercially available software products such as Informatica (www.informatica.com); Sagent (www.sagenttech.com); and/or DataStage (www.ascentialsoftware.com). The purpose of the ETL Engine is to extract, transform and load data from disparate sources into a new integrated physical data store. Atomic data from disparate sources may be aggregated and manipulated for faster performance (queries). Using XML messaging infrastructure, integrated data may also be exchanged among disparate applications. The ETL Tool is an optional component of the present invention.

The metadata repository is the container for managing enterprise metadata. The metadata repository should conform to industry standards and provide the ‘glue’ that drives interoperability among applications. By exposing and interchanging metadata, disparate information systems may be loosely coupled without re-building new data stores. Metadata will be stored and exchanged via industry standards, such as XML Metadata Interchange (“XMI”). Metadata will essentially be the key to the driven web services of the present invention.

The Universal Description, Discovery and Integration (“UDDI”) project is a sweeping industry initiative that creates a platform-agnostic, open framework for describing services, discovering businesses, and integrating business services using the Internet, as well as an operational registry. UDDI is the first truly cross-industry effort driven by all major platform and software providers, as well as marketplace operators and e-business leaders. These technology and business pioneers are acting as the initial catalysts to quickly develop UDDI and related technologies. UDDI may also be implemented within an organization to describe and expose services inside the firewall (intranet). Depending upon the eventual selection of the metadata repository, UDDI repository may also be implemented as a part of the metadata repository. Metadata repository will manage XML DTD's and/or Schemas.

Unlike the ETL Tools that are often used to create an integrated physical data store, the Virtual Data Access Engine is used to create ‘virtual’ views of data from disparate sources. This layer may be viewed as a ‘virtual mapping’ or a ‘roadmap’ to the underlying data sources that may be integrated at run-time and provide ‘context rich’ views of disparate data. Xaware's (www.xaware.com) or Metamatrix's Integration Server (www.metamatrix.com) or GoXML's integration server (www.goxml.com) may be used for this functionality. Disparate data sources will be modeled in the metadata repository as ‘virtual models’ (UML models) including run-time (database connectivity, query optimization information) metadata. The integration server will consume this information to direct queries to data sources and aggregate data as necessary.

In order to connect to data sources that may reside in relational and non-relational sources, software vendors have developed “Adapters” (software modules) that facilitate connectivity to data. These include ODBC, JDBC and native drivers to relational databases like Oracle, Sybase, DB2 and others. Custom adapters (if necessary) shall be developed although an extensive range of commercially available Adapters is already available and being used in most IT organizations. A Connector Development Kit will be provided to develop any specialized connector.

For example, in the life sciences industry, one question that may come up in data analysis is “What kind of chemical structures have been proposed for this disease?” and “What drugs have proven effective with these structures and which have adverse side effects?” The system of the present invention will generate a web service query that will search the respective Chemical Libraries, Bioassay, Human Genome Sequence, Proteomics databanks and Clinical/Pre-clinical trials databases and retrieve a results set. Additional data transformation and aggregation may then be performed by the researcher before sharing these results or performing another web service query.

The present invention can also be used to provide a “patent filing web service.” This service will automate the process of patent filing including searching and providing additional information requested (Toxicology/Adverse impact analysis data for example). The present invention may also include specialized web services such as patent preparation/submission, hooks (via web services) into industry (e.g., hospitals, business or government data stores), and for the healthcare industry such things as disease outcomes and diagnostic codes data.

The architecture provided by the present invention is integrated (ability to generate disparate sources and types of metadata), scalable (ability to sustain growth (content and usability of metadata)), robust (provide extensive functionality and performance), customizable (ability to tailor the metadata solution to satisfy the content complexity and business needs), open (accessibility of metadata to systems, applications and user interfaces), conformant with industry standards (ability to implement established industry metadata standards: MOF, CWM and XMI for example), bi-directional (permit metadata exchange (update) between the metadata sources and metadata repository) and closed-loop (allow metadata repository to feed metadata back to operational systems). The components described above in system 100 may be variants of commercial available metadata repository products:

| Meta- | | | |
|---|---|---|---|
| Nome | | | |
| Comp- | Technology | | |
| onent | Vendors | URL | Comments |
| MetaLife | Xaware Inc. | www.xaware.com | Xaware can |
| Pre- | MetaIntegration | www.metaintegration.net | provide |
| Processor | Inc. | | adapters and |
| | | | connectors for |
| | | | data sources, |
| | | | ERPs/CRM |
| | | | solutions. |
| | | | Meta- |
| | | | Integration |
| | | | can provide |
| | | | metadata |
| | | | interchange |
| | | | bridges. |
| MetaLife | Barnhill | No URL at this time | Barnhill |
| Cataloger/ | Genomics | Genomics for | |
| Classifier | Pavilion | www.pavtech.com | SVM software. |
| | Technologies | Other vendors | |
| | PrudSys | www.prudsys.com | have different |
| | X-Mine | www.x-mine.com | NN/SVM/DM |
| | | | technologies. |
| MetaLife | Ontogenics Corp. | www.ontogenics.com/ | |
| Modeler | Metanology Corp. | www.metanology.com | |
| | Adaptive Inc. | www.adaptive.com | |
| MetaLife | Adaptive Inc. | www.adaptive.com | Metadata |
| Repository | ASG | www.asg.com/ | Repository |
| | MetaMatrix Inc. | www.metamatrix.com | providers |
| | MetaIntegration | www.metaintegration.net | |
| | Inc. | | |
| MLIS | Xaware Inc. | www.xaware.com | |
| | MetaMatrix Inc. | www.metamatrix.com | |
| | MetaIntegration | www.metaintegration.net | |
| | Inc. | | |
| MetaLife | Adaptive Inc. | www.adaptive.com | |
| Portal | | | |

The commercially available components listed above cannot be taken “off the shelf” and combined together to create system 100 for life sciences without special modifications. The present invention provides an integrated system that is not currently available.

The MetaLife Repository supports numerous industry standards. The supported 15 standards from the Object Management Group include Meta Object Facility (“MOF”), XML Metadata Interchange (“XMI”), Unified Modeling Language (“UML”), Common Warehouse MetaModel (“CWM”), Software Process Engineering MetaModel (“SPEM”), Component Collaboration Architecture (“EDOC CCA”), and Software Portfolio Management Facility (“SPMF”). Supported life sciences domain standards includes gene expression, genome maps, clinical image access service, lab instrument control interface, and biomolecular sequence analysis. Life sciences markup languages and ontologies are also supported. In addition, the Reusable Asset Specification (“RAS”) and Java Metadata Interface (“JMI”) are supported.

FIG. 2 is a block diagram of a system 200 in accordance with another embodiment of the present invention. The system 200 includes a MetaLife Classifier 104, a MetaLife Modeler 106, a MetaLife Repository 108, a MetaLife Pre-Processor 110 and a MetaLife Portal 112. The components are the same as described in FIG. 1, except that they are connected differently.

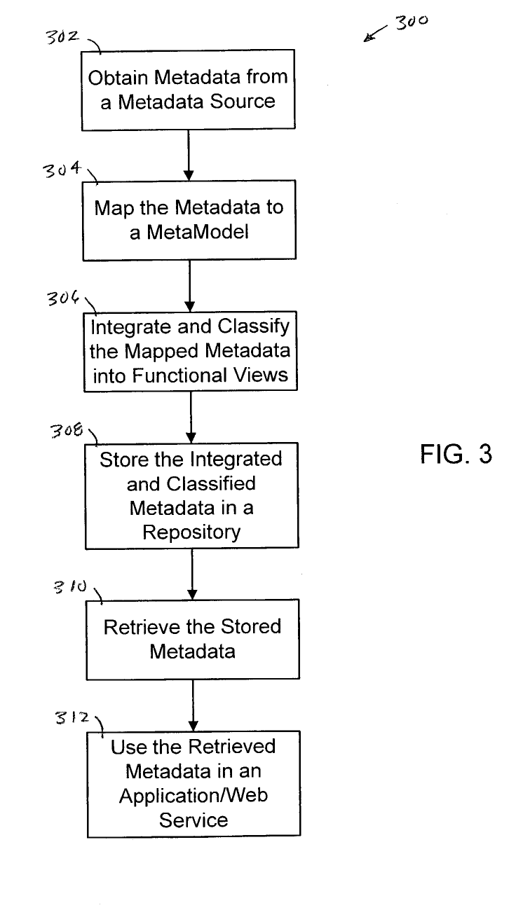

FIG. 3 is a flow chart of a method 300 in accordance with one embodiment of the present invention. The method 300 obtains metadata from a metadata source in block 302. Thereafter, the metadata is mapped to a MetaModel in block 304 and the mapped metadata is integrated and classified into functional views in block 306. The integrated and classified metadata is then stored in a repository in block 308. The stored metadata is retrieved in block 310 and used in an application/web service in block 312.

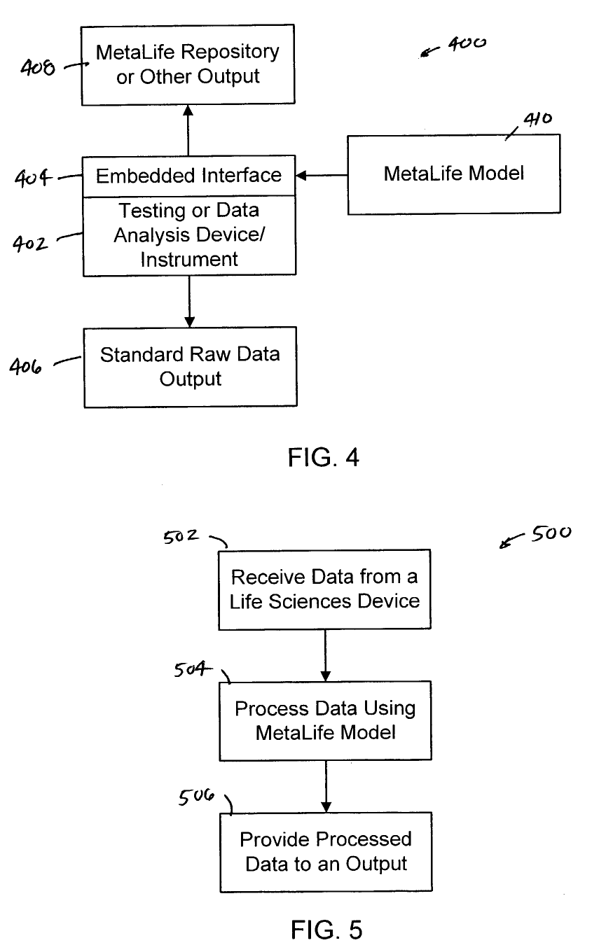

FIG. 4 is a block diagram of a system 400 in accordance with another embodiment of the present invention. The system 400 includes a testing or data analysis/instrument device 402 having an embedded interface 404. The testing or data analysis/instrument device 402 produces a standard raw data output 406. In addition, the metadata from the testing or data analysis/instrument device 402 is processed or consumed by the embedded interface 404 using a MetaLife Model 410, which can be downloaded from a MetaLife Repository. The output data is then provided to a MetaLife Repository or other selected output 408, such as an XML file or another device.

FIG. 5 is a flow chart of a method 500 in accordance with another embodiment of the present invention. The method 500 corresponds to the system 400 (FIG. 4). Specifically, the Embedded Interface 404 receives the data from the Testing or Data Analysis/Instrument Device 402 in block 502 and processes or consumes that data using the MetaLife Model 410 in block 504. Thereafter, the processed data is provided to a MetaLife Repository or other output device/application 408 in block 506.

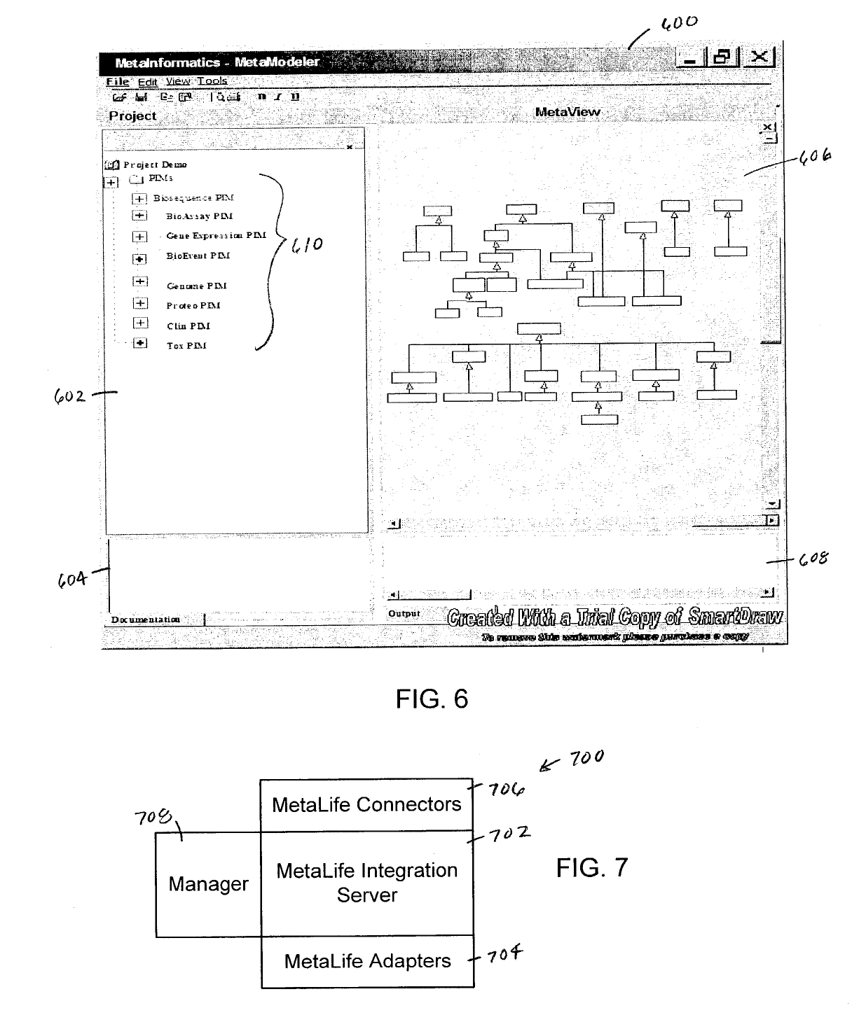

FIG. 6 is a screen shot 600 of a MetaLife Modeler 106 (FIGS. 1 and 2) in accordance with one embodiment of the present invention. The MetaLife Modeler is a graphical user interface that enables metadata modeling conformant to OMG's Model Driven Architecture (“MDA”) using UML. The MetaLife Modeler allows abstraction of metadata at design time and run time using semantics and business rules. The MetaLife Modeler permits complete integration and exchange of metadata with existing modeling tools, such as ETL and DW, via XML. The MetaLife Modeler also allows complete modeling of web services/application as well as more than 90% of the code generation. The screen 600 is split into a project window 602, documentation window 604, model window 606 and output window 608. The project window 602 lists the various models 610, such as biosequence, bioassay, gene expression, bioevent, genome, proteomic, clinical trial and toxicology models, that are available in a standard file-tree structure. Once selected, the various models 610 can be displayed in the model window 606 and manipulated. The MetaLife Modeler promotes understanding of business needs, satisfies questions, provides focus on important issues, removes ambiguity, tests ideas, compares alternatives, provides rigor, reduces cost of changes and corrections, and supports new iterations.

FIG. 7 is a block diagram of a MetaLife Integration Server 700 in accordance with one embodiment of the present invention. The MetaLife Integration Server 700 provides bi-directional integration of disparate enterprise systems. The MetaLife Integration Server 700 also can decompose XML data to enterprise system, manage transactions across systems, apply business rules, workflow logic and transformations to data, aggregate data from disparate systems to create virtual business objects, and reuse semantic accuracy of enterprise metadata. The MetaLife Integration Server 700 includes a MetaLife Integration Server 702 communicably coupled to one or more MetaLife Adapters 704, one or more MetaLife Connectors 706 and a manager 708. The MetaLife Integration Server 702 is a XML based bi-directional server (Java and C++) that can be deployed on J2EE servers and .Net servers, Windows and Unix platforms. The MetaLife Adapters 704 connect the MetaLife Integration Server 702 to enterprise systems, such as RDBMS, XML, DBMS, HTTP, EJB's, JMS, Java, API, SOAP, mainframe, ERP, CRM, SNMP and SOCKET. The MetaLife Connectors 706 connect other applications to the MetaLife Integration Server 702, such as XQUERY, EJB, JMS, SERVLET, SOAP, CGI, ISAPI, CORBA, HTTP and API. The Manager 708 manages the MetaLife Integration Server 702.

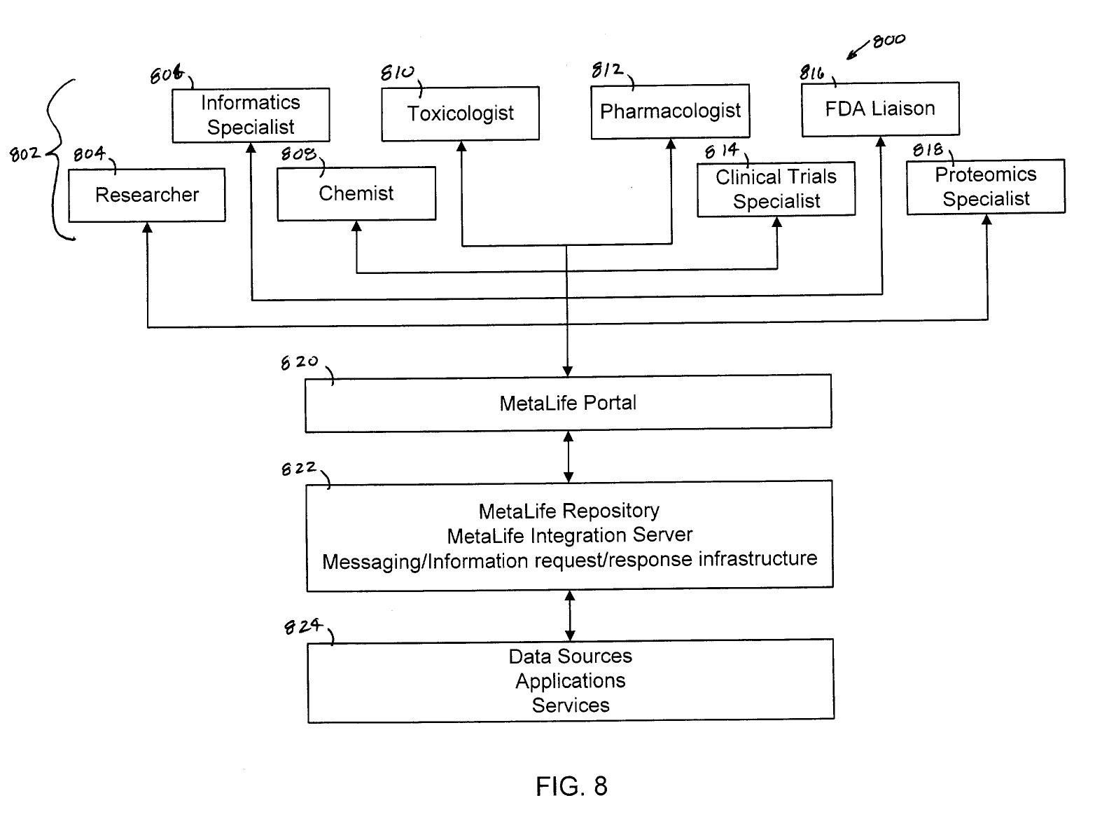

FIG. 8 is a block diagram of a system 800 in accordance with another embodiment of the present invention. The system 800 includes three tiers: a MetaLife access tier 820, a data storage and processing tier 822 and a data source tier 824. Various users 802 use the access tier 820, which includes the MetaLife Portal, to access and use and manipulate metadata that is stored or accessible via the data storage and processing tier 822. The various users 802 may include researchers 804, informatics specialists 806, chemists 808, toxicologists 810, pharmacologists 812, clinical trials specialists 814, FDA liaisons 816, proteomics specialists 818 and others. The data storage and processing tier 822 includes the MetaLife Repository (software services/applications directory), the MetaLife Integration Server, and the messaging/information request/response infrastructure. The data source tier 822 includes internal and external data sources, internal and partner applications, and internal and external services.

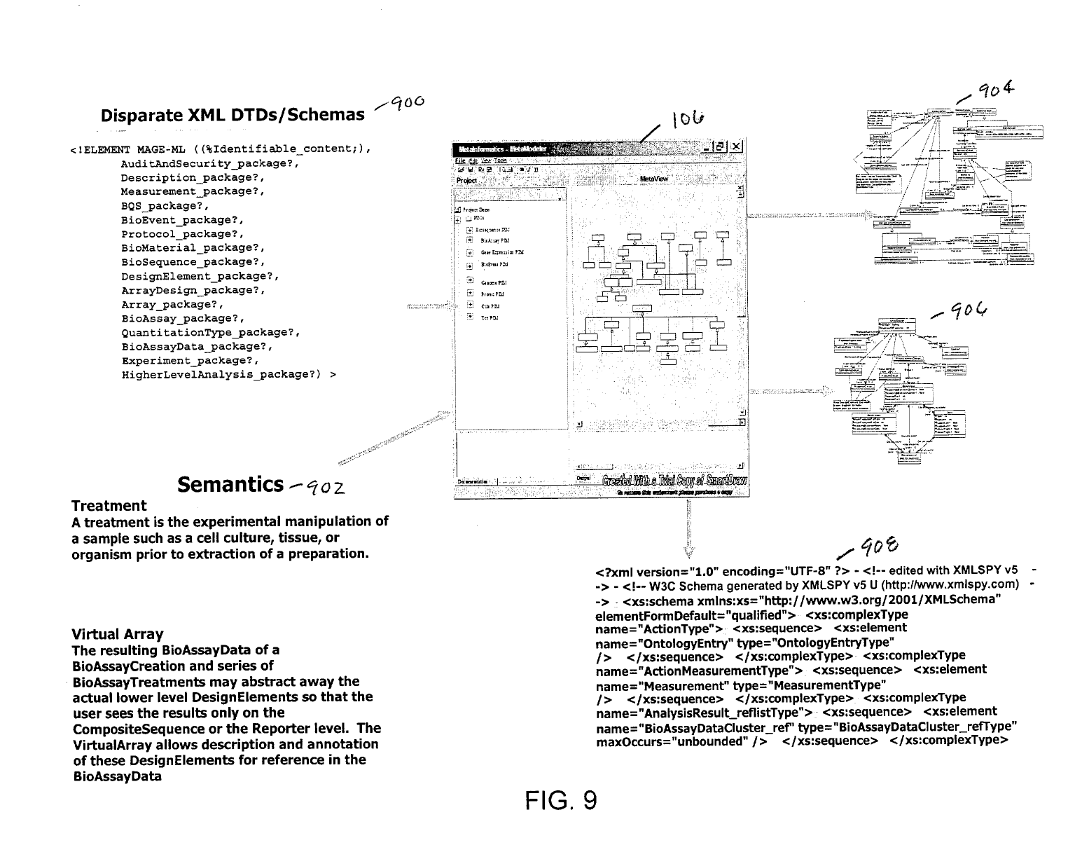

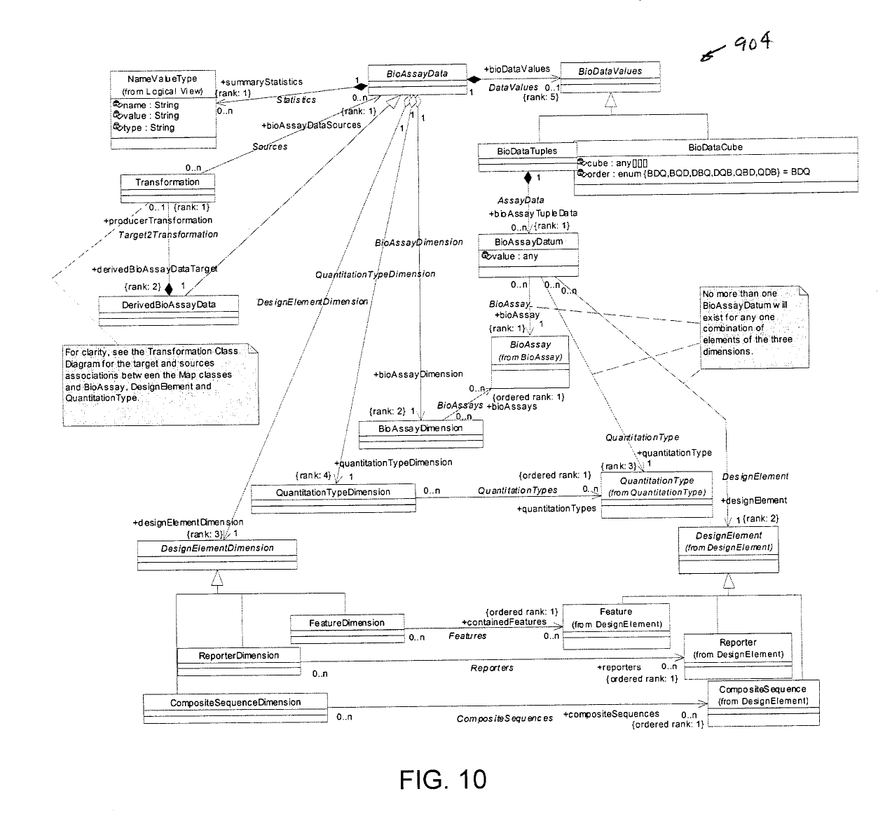

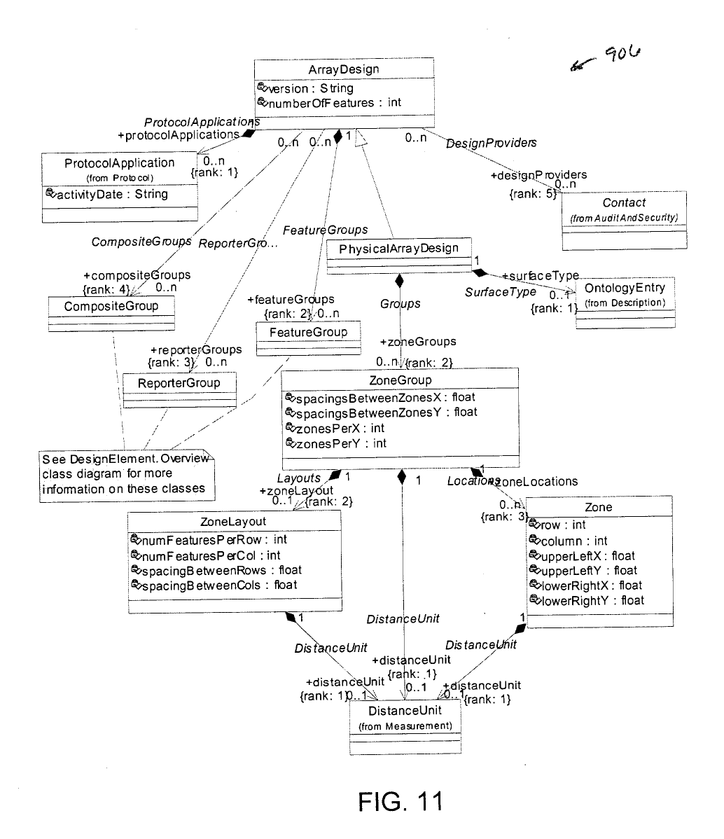

FIG. 9 is a diagram illustrating the uses of the MetaLife Modeler 106 (FIGS. 1 and 2) in accordance with one embodiment of the present invention. As shown, the MetaLife Modeler 600 allows the user to create and manipulate MetaModels using disparate XML DTDs/Schemas 900, Semantics 902, MetaModels 904 and 906, and MetaModel output 908. For example, the Semantics 902 may include a treatment, which is the experimental manipulation of a sample such as a cell culture, tissue, or organism prior to extraction of a preparation, or a virtual array, which is the resulting BioAssayData of a BioAssayCreation and series of BioAssayTreatments may abstract away the actual lower level design elements so that the user sees the results only on the composite sequence or the reporter level. The virtual array allows description and annotation of these design elements for reference in the BiaAssayData. MetaModel 904 is a model for BioAssayData and is shown in more detail in FIG. 10. MetaModel 906 is a model for ArrayDesign and is shown in more detail in FIG. 11.

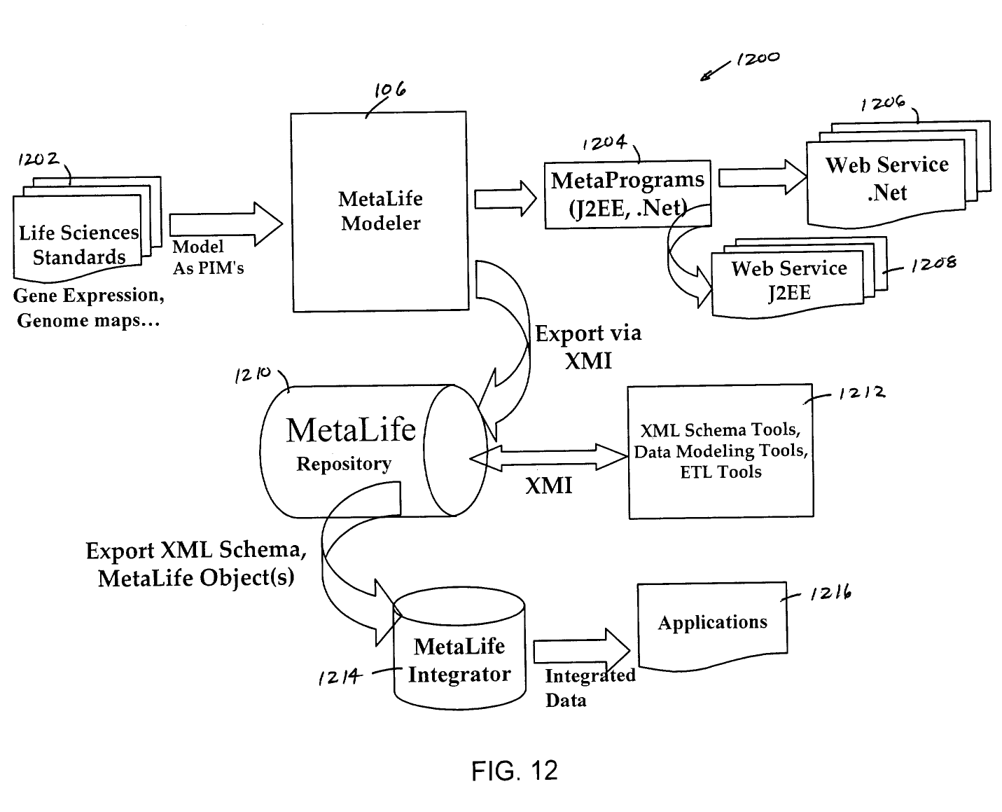

FIG. 12 is a block diagram of a data flow 1200 in accordance with one embodiment of the present invention. Life sciences standards 1202, such as gene expression and genome maps, are modeled as PIM's in a MetaLife Modeler 106 (FIGS. 1 and 2). The MetaModels can then be used in MetaPrograms (J2EE or .Net) 1204 to provide .Net web services 1206 and J2EE web services 1208. The MetaModels can also be exported via XMI to the MetaLife Repository 1210. The Metadata and MetaModels in the MetaLife Repository 1210 may then be used by various tools 1212, such as XML Schema Tools, Data Modeling Tools and ETL Tools, via XMI. XML Schema and MetaLife Object(s) may also be exported from the MetaLife Repository 1210 to the MetaLife Integrator 1214, which, in turn, provides integrated data to applications 1216.

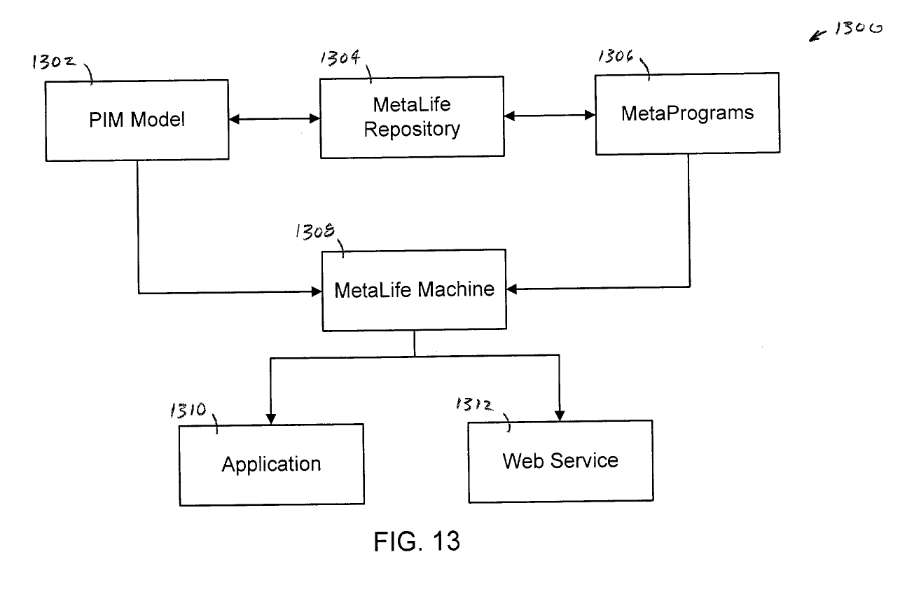

FIG. 13 is a block diagram of a system 1300 in accordance with another embodiment of the present invention. System 1300 is used to generate applications 1310 and web services 1312. The PIM Model 1302 uses UDDI, WSDL, SOAP and XML Schemas in the MetaLife Repository 1304 to provide a MetaModel to the MetaLife Machine 1308. The MetaLife Repository 1304 is also used to generate MetaPrograms 1306, which are applied to the MetaLife Machine 1308. The MetaLife Machine 1308 then generates code to produce applications 1310 (J2EE or Net) and web services 1312.

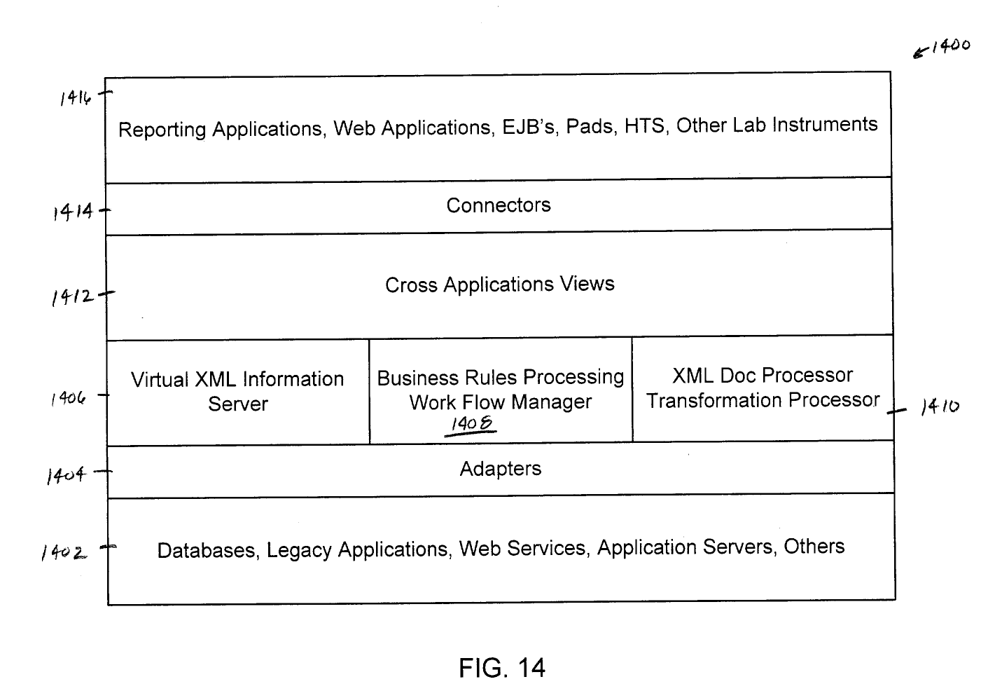

FIG. 14 is a block diagram of a MetaLife Integration Server 1400 in accordance with another embodiment of the present invention. The first tier 1402 contains databases, legacy applications, web services, application servers and other data sources. The second tier 1404 contains adapters 1404 that are used to process metadata from the first tier to the third tier 1406, which contains a virtual XML information server 1406, business rules processing and work flow manager 1408, and XML doc processor and transformation processor 1410. The third tier 1406 works with the fourth tier 1412, which contains cross applications views, to provide metadata integration. The fifth tier 1414 contains connectors that are used to supply integrated metadata to the sixth tier, which includes reporting applications, web applications, EJB's, Pads, HTS and other lab instruments.

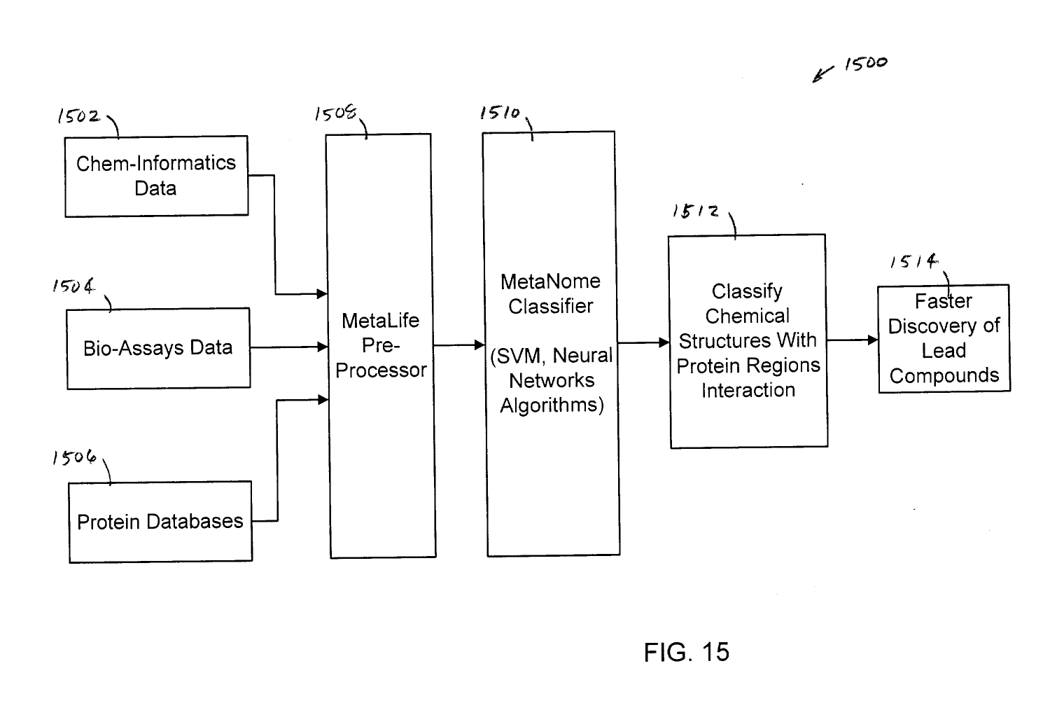

FIG. 15 is a block diagram of a data flow 1500 in accordance with another embodiment of the present invention. Data flow 1500 illustrates the prediction of highly effective chemical compounds, gene and protein structures for drug discovery, diagnostics and improvement of the HTS process. Chem-informatics data 1502, bio-assays data 1504 and protein databases 1506 are fed to the MetaLife Pre-Processor 1508. The MetaLife Pre-Processor 1508 provides pre-processed metadata to the MetaLife Classifier 1510, which may include SVM or Neural Network algorithms. Chemical structures are then classified with protein regions interaction 1512 to produce faster discovery of lead compounds 1514.

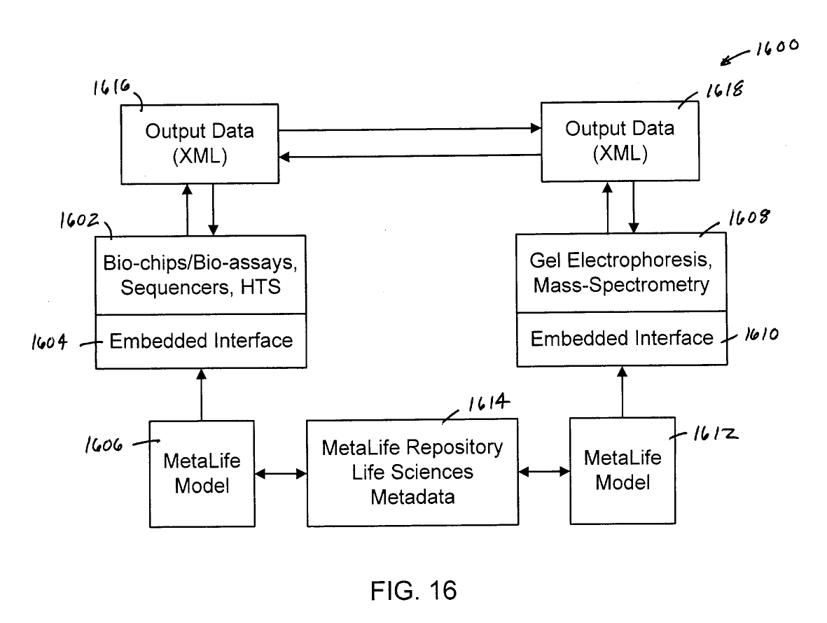

FIG. 16 is a block diagram of a system 1600 in accordance with another embodiment of the present invention. The present invention provides device driven interoperability by creating output data that can be bi-directionally exchanged between devices. A first testing or data analysis/instrument device 1602, such as Bio-chips, Bio-assays, sequencers or HTS, has a first embedded interface 1604. The first testing or data analysis/instrument device 1602 uses the first embedded interface 1604 to produces first output data 1616, which may be in XML. The first embedded interface 1604 processes or consumes the metadata generated by the first testing or data analysis/instrument device 1602 using a MetaLife Model 1606, which may be downloaded from MetaLife Repository 1614. Similarly, a second testing or data analysis/instrument device 1608, such as gel electrophoresis or mass-spectrometry, has a second embedded interface 1610. The second testing or data analysis/instrument device 1608 produces second output data 1618, which may be in XML. The second embedded interface 1610 processes or consumes the metadata generated by the second testing or data analysis/instrument device 1608 using a MetaLife Model 1612, which may be downloaded from MetaLife Repository 1614.

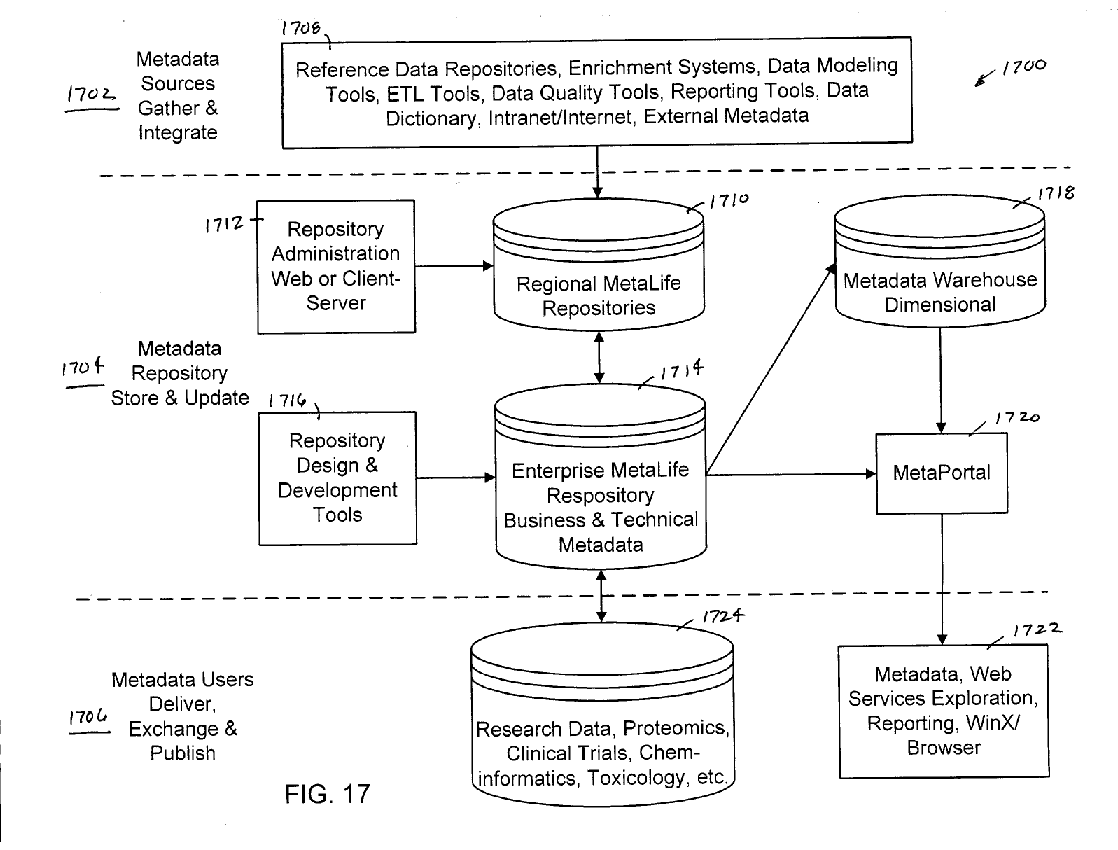

FIG. 17 is a block diagram of a system 1700 in accordance with another embodiment of the present invention. The system 1700 includes Metadata sources 1702, which are used to gather and integrate metadata, a Metadata Repository 1704, which is used to store and update metadata, and Metadata Users 1706, which deliver, exchange and publish metadata. The Metadata sources 1702 include such sources 1708 as reference data repositories, enrichment systems, data modeling tools, ETL Tools, data quality tools, reporting tools, data dictionary, intranet/internet and external metadata. The Metadata Repository 1704 includes regional MetaLife Repositories 1710, repository administration web or client server 1712, enterprise MetaLife Repository 1714, repository design and development tools 1716, Metadata warehouses 1718 and MetaPortal 1720. Metadata sources 1708 are communicably coupled to regional Metadata Repositories 1710. The Metadata Users 1706 includes metadata, web services exploration, reporting, WinX/Browser 1722 and research data, proteomics, clinical trials, cheminformatics, toxicology, etc. 1724. The regional MetaLife Repositories 1710 are communicably coupled to repository administration web or client server 1712 and enterprise MetaLife Repository 1714. Enterprise MetaLife repository 1714, which contains business and technical metadata, is communicably coupled to repository design and development tools 1716, Metadata warehouses 1718, MetaPortal 1720 and reference data, research data, clinical trials, cheminformatics and toxicology 1724. The MetaPortal 1722 is also communicably coupled to the Metadata warehouse 1718 and the Metadata, web services exploration, reporting, WinX/Browser 1722.

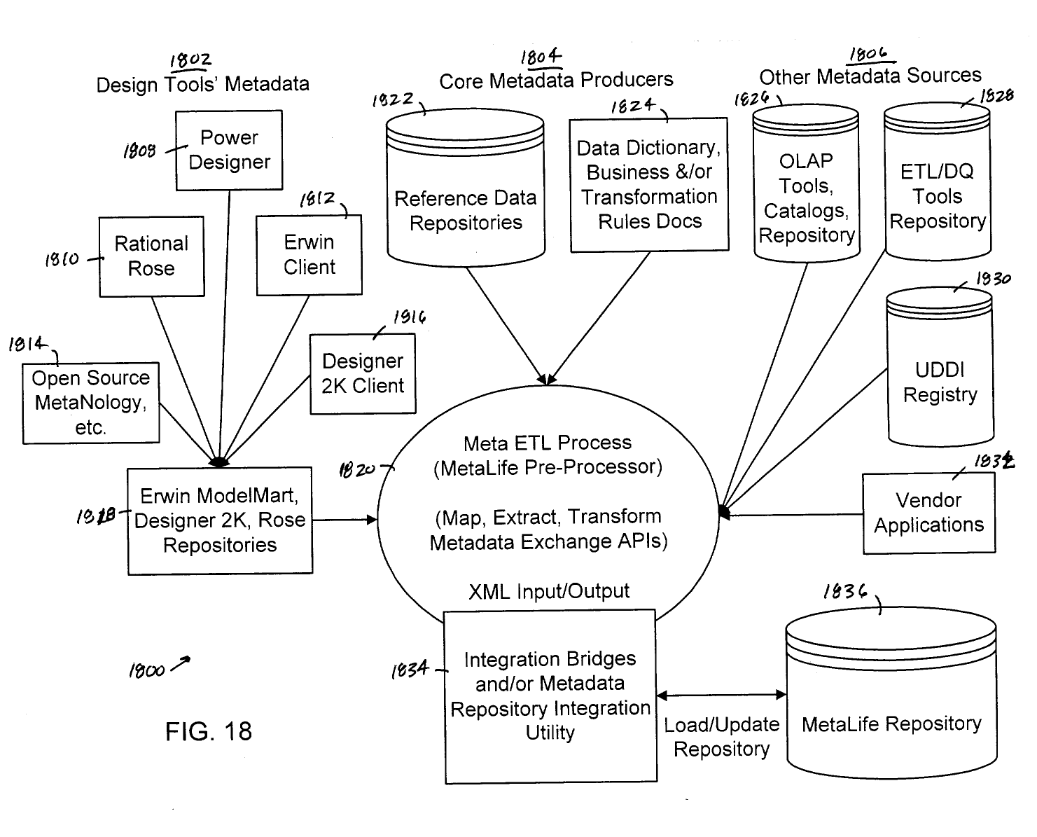

FIG. 18 is a block diagram of a system 1800 in accordance with another embodiment of the present invention. System 1800 includes design tools Metadata 1802, core Metadata producers 1804 and other Metadata sources 1806. The design tools Metadata 1802 includes Power Designer 1808, Rational Rose 1810, Erwin Client 1812, Open Source (MetaNology, etc.) 1814 and Designer 2K Client 1816 all communicably coupled to the Erwin, ModelMart, Designer 2K and Rose repositories 1818, which are communicably coupled to the Meta ETL Process 1820. The core Metadata producers 1804 include reference data repositories 1822, and data dictionary, business and/or transformation rules does 1824, each communicably coupled to the Meta ETL process 1820. The other Metadata sources 1806 include OLAP tools, catalogs and repositories 1826, ETL/DQ tools repository 1828, UDDI registry 1830 and vendor applications 1832, each communicably coupled to the Meta ETL process 1820. The Meta ETL process (MetaLife Pre-Processor) 1820 maps, extracts, transforms using Metadata exchange APIs to provide XML input/output. The Meta ETL process 1820 is communicably coupled to the integration bridges and/or Metadata repository integration utility 1834. The integration bridges 1834 are communicably coupled to the MetaLife repository 1836 to load and update the repository information.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method for using life sciences metadata comprising the steps of:
obtaining the metadata from a metadata source;
mapping the metadata to a metamodel;
integrating and classifying the mapped metadata into functional views;
storing the integrated metadata in a repository;
retrieving the stored metadata; and
using the retrieved metadata in one or more applications.

2. The method as recited in claim 1, wherein the metamodel is obtained from an industry standard specification for life sciences.

3. The method as recited in claim 1, wherein the one or more applications includes one or more web services.

4. The method as recited in claim 3, wherein the web service searches the respective Chemical Libraries, Bioassay, Human Genome Sequence, Proteomics databanks and Clinical/Pre-clinical trials databases and retrieve a results set.

5. The method as recited in claim 1, further comprising the step of transforming additional data to generate a web service query that will search the respective Chemical Libraries, Bioassay, Human Genome Sequence, Proteomics databanks and Clinical/Pre-clinical trials databases and retrieve a results set.

6. The method as recited in claim 1, further comprising the step of aggregating data to generate a web service query that will search the respective Chemical Libraries, Bioassay, Human Genome Sequence, Proteomics databanks and Clinical/Pre-clinical trials databases and retrieve a results set.

7. The method as recited in claim 6, further comprising the step of transforming and aggregating the data and sharing the results.

8. The method as recited in claim 6, further comprising the step of transforming and aggregating the data and sharing the results and performing another web service query.

9. A computer program embodied on a computer readable medium for using life sciences metadata comprising:
a code segment for obtaining the metadata from a metadata source;
a code segment for mapping the metadata to a metamodel;
a code segment for integrating and classifying the mapped metadata into functional views;
a code segment for storing the integrated metadata in a repository;
a code segment for retrieving the stored metadata; and
a code segment for using the retrieved metadata in one or more applications.

10. The computer program as recited in claim 9, wherein the metamodel is obtained from an industry standard specification for life sciences.

11. The computer program as recited in claim 9, wherein the one or more applications includes one or more web services.

12. The computer program as recited in claim 11, wherein the web service searches the respective Chemical Libraries, Bioassay, Human Genome Sequence, Proteomics databanks and Clinical/Pre-clinical trials databases and retrieve a results set.

13. The computer program as recited in claim 9, further comprising a code segment for transforming additional data to generate a web service query that will search the respective Chemical Libraries, Bioassay, Human Genome Sequence, Proteomics databanks and Clinical/Pre-clinical trials databases and retrieve a results set.

14. The computer program as recited in claim 9, further comprising a code segment for aggregating data to generate a web service query that will search the respective Chemical Libraries, Bioassay, Human Genome Sequence, Proteomics databanks and Clinical/Pre-clinical trials databases and retrieve a results set.

15. The computer program as recited in claim 14, further comprising a code segment for transforming and aggregating the data and sharing the results.

16. The computer program as recited in claim 14, further comprising a code segment for transforming and aggregating the data and sharing the results and performing another web service query.

17. A system for semantic metadata processing comprising:
a MetaLife portal;
a MetaLife modeler;
a MetaLife integration server; and
a MetaLife repository communicably coupled to the MetaLife portal, the MetaLife modeler and the MetaLife integration server.

18. The system as recited in claim 17, further comprising a MetaLife classifier communicably coupled to the MetaLife repository.

19. The system as recited in claim 18, further comprising a MetaLife pre-processor communicably coupled to the MetaLife classifier.

20. A system for semantic metadata processing comprising:
a MetaLife modeler;
a MetaLife pre-processor communicably coupled to the MetaLife modeler; and
a MetaLife repository communicably coupled to the MetaLife modeler.

21. The system as recited in claim 20, further comprising a MetaLife portal communicably coupled to the MetaLife repository.

22. The system as recited in claim 20, further comprising a MetaLife classifier communicably coupled to the MetaLife repository, the MetaLife modeler and the MetaLife pre-processor.

23. A system for integrating and analyzing life sciences data from one or more data sources comprising:
a metadata repository;
a virtual data access engine communicably coupled to the metadata repository;
one or more adapters communicably coupled to the one or more data sources and the metadata repository; and
an integration server communicably coupled to the metadata repository that gathers information to direct queries to the one or more data sources, aggregates data received from the one or more data sources and provides an output file.

24. A system as recited in claim 23, further comprising an Extract, Transformation & Load Engine communicably coupled to the metadata repository.

25. The system as recited in claim 23, wherein the metadata repository is a UDDI Repository.

26. The system as recited in claim 23, wherein the integration server generates a web service query that searches the respective Chemical Libraries, Bioassay, Human Genome Sequence, Proteomics databanks and Clinical/Pre-clinical trials databases and retrieve a results set.

27. The system as recited in claim 23, wherein the integration server transforms additional data to generate a web service query that will search the respective Chemical Libraries, Bioassay, Human Genome Sequence, Proteomics databanks and Clinical/Pre-clinical trials databases and retrieve a results set.

28. The system as recited in claim 23, wherein the integration server aggregates data to generate a web service query that will search the respective Chemical Libraries, Bioassay, Human Genome Sequence, Proteomics databanks and Clinical/Pre-clinical trials databases and retrieve a results set.

29. The system as recited in claim 23, wherein the integration server transforms and aggregates the data and sharing the results.

30. The system as recited in claim 23, wherein the integration server transforms and aggregates the data, shares the results and performs another web service query.

31. A method for consuming metadata from a life sciences device comprising the steps of:
receiving data from the life sciences device;
processing the data using a MetaLife model; and
providing the data to an output.

32. A computer program embodied on a computer readable medium for consuming metadata from a life sciences device comprising:
a code segment for receiving data from the life sciences device;
a code segment for processing the data using a MetaLife model; and
a code segment for providing the data to an output.

33. A system comprising:
a life sciences device;
an interface embedded within the life sciences device; and
a MetaLife model loaded within the embedded interface.

34. The system as recited in claim 33, further comprising a MetaLife repository communicably coupled to the embedded interface.
